(19) United States
(12) Patent Application Publication
(10) Pub. No.: US 2018/0147556 A1
(43) Pub. Date: May 31, 2018

(54) BI-STRUCTURED MATRIX FOR SOLID REACTANTS PURIFICATION AND HANDLING AND METHODS FOR OBTAINING SAID MATRIX

(71) Applicants: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE QUILMES, Bernal Pcia. de Buenos Aires (AR); INIS BIOTECH LLC, Milford Kent County, DE (US)

(72) Inventors: Mirna SANCHEZ, Quilmes Oeste Pcia. de Buenos Aires (AR); Mariano GRASSELLI, Ciudad Autónoma de Buenos Aires (AR); Leandro J. MARTINEZ, Quilmes Pcia. de Buenos Aires (AR)

(73) Assignees: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE QUILMES, Bernal Pcia. de Buenos Aires (AR); INIS BIOTECH LLC, Milford Kent County, DE (US)

(21) Appl. No.: 15/534,872

(22) PCT Filed: Dec. 11, 2015
(86) PCT No.: PCT/IB2015/059557

§ 371 Date: Jun. 9, 2017

(30) Foreign Application Priority Data
Dec. 11, 2014 (AR) 20140104604

(51) Int. Cl. B01J 20/26; B01L 3/02; C12N 15/10; C07K 1/16; B01J 20/28; B01J 20/24; B01J 20/10; B01J 20/32

(52) U.S. Cl. CPC B01J 20/267; B01L 2300/16; C12N 15/101; C07K 1/16; B01J 20/28045; B01J 20/261; B01J 20/24; B01J 20/103; B01J 20/3291; B01J 20/3212; B01J 20/327; B01J 20/3225; B01J 20/3282; B01L 2200/16; B01L 2200/0631; B01L 2200/12; B01L 3/0275

(57) ABSTRACT

Bi-structured matrix for purification and handling of solid reagents, which comprises at least a polymer solid carrier coated with at least one hydrosoluble polymer, and manufacturing processes. The solid carrier may be, among others, cross-linked polyurethane foam or a micropipette tip. The hydrosoluble polymer may be, among others, polyvinylalcohol, agarose, hydroxyethylcellulose or combinations thereof. The matrix may further comprise a polymer produced from monomers of glycidyl metacrylate (GMA), dimethyl acrylamide (DMAAm), 2-hydroxyethyl metacrylate, metacrylic acid, or combinations thereof.

The present invention refers to a bi-structured matrix for purification and handling of solid reactants that comprises at least a polymer solid carrier coated with at least a hydrosoluble polymer and methods for obtaining said matrix. The solid carrier may be, among others, cross-linked polyurethane foam or a micropipette tip. The hydrosoluble polymer may be, among others, polyvinyl alcohol, agarose, hydroxyethyl cellulose or combinations thereof. The matrix may further comprise glycidyl methacrylate (GMA), dimethyl acrylamide (DMAAm), 2-hydroxyethyl methacrylate, methacrylic acid, or combinations thereof.

### BACKGROUND OF THE INVENTION

Modern biotechnology largely depends on availability of materials that allow obtaining competitive products regarding quality and costs. New materials based on polymers allow the development of novel, more sensitive, faster analysis techniques which require a lower amount of sample.

There is a wide range of polymers with different physical and chemical properties, which are also inexpensive. Generally, there are two kinds of polymers, solid polymers, whose main function is mechanical, kind substance containers, especially when handling liquids; and hydrogel-type polymers with specific functional properties. Examples of laboratory products formed by polymers are automatic micropipette tips and all kind of containers for handling and keeping liquids, solids and biological material (Falcon® and Eppendorf® tubes, ELISA plates, etc.)

Hydrogels use has became widespread in various biological areas, such as contact lens materials, matrixes for cell encapsulation and devices for controlled release of medicines (Vinogradov et al., 2002; Hoffman et al., 2002; Casolaro et al., 2006 and Bae et al., 2006).

For the last two decades, one-piece (monolithic) porous polymeric materials have been widely researched for potential applications in macromolecules separation (Park et al., 2013). These materials have two additional properties: (i) a mechanical structure and (ii) an internal structure with interconnected channels that facilitate mass transport (Zhang et al., 2001; Cabrera et al., 2000). Therefore, monolithic columns with these single structures allow high flow rates under low pressure without loss of column efficiency, resulting in a fast separation (Yu et al., 1999).

There is a wide variety of techniques that allow to prepare monolithic porous solids, disclosed by Svec (Svec et al., 2010). A method for preparing said materials is through a polymerization reaction under low temperature (cryogenic) conditions. These materials are called “cryogels” because of the preparation method thereof (Mattiasson et al., 2009).

Most common solid polymers, like polyethylene, polystyrene and polypropylene, have good chemical stability (desirable property) but very harsh conditions are required for their modification.

Polymer modification techniques by ionizing radiation are a powerful methodology for preparing new materials, given its simplicity and low demand of special chemical reactants and solvents. Currently, said techniques are applied to the production of mass-use products, liken car tires or any kind of polymeric cables and tubes. The two more common applications are in cross-linking reactions, which improve mechanical properties of materials, and degradation reactions that reduce polymer molecular weight. There is also a third alternative technique, less often employed, which allows to obtain new materials by combining ionization reactions and free radical polymerization, rendering modified materials by grafting in different proportions and locations of the base material (Kobayashi et al., 1993; Ventura et al., 2008).

Solid polymers are generally hydrophobic, with a low compatibility with water soluble polymers. Classic chemical reactions of modification by wet process have a very low yield, modifying only the first superficial molecular layers of the materials. Besides, said reactions produce a large amount of toxic waste that has to be adequately disposed of.

In recent years, hydrogel production induced by ionizing radiation has been a very active research area. A great effort in R&D in super-absorbent hydrogels for soil remediation is currently underway (IAEA Tecdoc, 2014). A key aspect for successful intermolecular cross-linking of hydrophilic polymers, especially polysaccharides, is the amount of liquid present during the irradiation step. As an example, hydrophilic polymer carboxymethyl cellulose is degraded by ionizing radiation if it is irradiated in dry form or as a diluted aqueous solution. However, it can turn cross-linked when it is irradiated as a paste prepared with water (Fei et al., 2000). Therefore, not only the different employed reactants are important, but also sample irradiation and preparation conditions are a critical aspect for obtaining the desired material.

Polymer ionizing radiation processing is a polymer modification industrial technique. The high penetration of electron beam and gamma radiation is capable of generating reactive radicals in the whole irradiated material in the first microseconds. Afterwards, said radicals are involved in different chemical reactions according to the sample composition and physical state, like physical distribution of polymers, monomers and dissolvent in the sample. Besides, relative molecular motility of radicals will allow different chemical reactions to occur, e.g. cross-linking, chemical bond breaking (chemical degradation) o radical-initiated polymerization (RIIP) in case that vinyl monomers exist in the medium.

### SUMMARY OF THE INVENTION

The present invention provides a bi-structured matrix for solid reactants purification and handling, comprising at least a polymeric solid carrier coated with at least a hydrosoluble polymer. The solid carrier may be, among others, cross-linked polyurethane foam or a micropipette tip. The hydrosoluble polymer may be, among others, polyvinyl alcohol, agarose, hydroxyethyl cellulose, or combinations thereof. The matrix may further comprise glycidyl methacrylate (GMA), dimethyl acrylamide (DMAAm), 2-hydroxyethyl methacrylate, methacrylic acid, or combinations thereof. In a preferred embodiment, the matrix comprises a solid carrier of cross-linked polyurethane foam coated with a hydrosoluble polymer selected from the group consisting of polyvinyl alcohol, agarose, hydroxyethyl cellulose and glycidyl methacrylate monomers (GMA) linked to said hydrosoluble polymer. In another preferred embodiment, the matrix comprises a solid carrier of cross-linked polyurethane foam coated with a hydrosoluble polymer selected from the group consisting of polyvinyl alcohol, agarose, hydroxyethyl cellulose, glycicyl methacrylate monomers (GMA) linked to the hydrosoluble polymer and sulfonic groups linked to the glycidyl methacrylate monomers (GMA). In another preferred embodiment the matrix comprises a solid carrier of cross-linked polyurethane coated with a hydrosoluble polymer selected from the group consisting of polyvinyl alcohol, agarose, hydroxyethyl cellulose, glycidyl methacrylate monomers (GMA) linked to the hydrosoluble polymer and iminodiacetic acid (IDA) linked to the glycidyl methacrylate monomers (GMA). In another preferred embodiment, the matrix comprises a pipette tip as solid carrier coated inside with a hydrosoluble polymer selected from the group consisting of polyvinyl alcohol, agarose and hydroxyethyl cellulose. In another preferred embodiment, the matrix comprises a pipette tip as solid carrier coated inside with hydrosoluble polymer selected from the group consisting of polyvinyl alcohol, agarose, hydroxyethyl cellulose, and silica particles.

The invention provides a process for preparing a matrix comprising the following steps: a) contacting a polymer solid carrier with at least a hydrosoluble polymer until obtaining a polymer solid carrier coated with a hydrosoluble polymer; b) drying the solid carrier coated with the hydrosoluble polymer and immersing the same into an irradiation solution; and c) irradiating with a 60-cobalt source and drying the obtained bi-structured matrix. The process may also comprise after step a) another step where the solid carrier coated with the hydrosoluble polymer is immersed in a coagulant selected from the group consisting of 2-propanol, ethanol, 1-propanol and dioxane.

The invention provides a process for preparing a matrix comprising the following steps: a) contacting a solid carrier of cross-linked polyurethane foam with a hydrosoluble polymer selected from the group consisting in polyvinyl alcohol, agarose and hydroxyethyl cellulose until obtaining a polymer solid carrier coated with a hydrosoluble polymer; b) immersing the solid carrier coated with the hydrosoluble polymer obtained in the previous step in a coagulant selected from the group consisting of 2-propanol, ethanol, 1-propanol and dioxane; c) drying the solid carrier coated with the hydrosoluble polymer from the previous step and immersing the same into an irradiation solution comprising glycidyl methacrylate monomers (GMA); and d) irradiating with a 60-cobalt source and drying the obtained bi-structured matrix.

The invention provides a process for preparing a matrix comprising the following steps: a) contacting a solid carrier of cross-linked polyurethane foam with a hydrosoluble polymer selected from the group consisting in polyvinyl alcohol, agarose and hydroxyethyl cellulose until obtaining a polymer solid carrier coated with a hydrosoluble polymer; b) immersing the solid carrier coated with the hydrosoluble polymer obtained in the previous step in a coagulant selected from the group consisting of 2-propanol, ethanol, 1-propanol and dioxane; c) drying the solid carrier coated with the hydrosoluble polymer from the previous step and immersing the same into an irradiation solution comprising glycidyl methacrylate monomers (GMA); d) irradiating with a 60-cobalt source and drying the obtained bi-structured matrix; and e) incubating the matrix obtained in the previous step with an aqueous solution comprising sodium sulfite and isopropanol, and drying the same.

The invention provides a process for preparing a matrix comprising the following steps: a) contacting a solid carrier of cross-linked polyurethane foam with a hydrosoluble polymer selected from the group consisting in polyvinyl alcohol, agarose and hydroxyethyl cellulose until obtaining a polymer solid carrier coated with a hydrosoluble polymer; b) immersing the solid carrier coated with the hydrosoluble polymer obtained in the previous step in a coagulant selected from the group consisting of 2-propanol, ethanol, 1-propanol and dioxane; c) drying the solid carrier coated with the hydrosoluble polymer from the previous step and immersing the same into an irradiation solution comprising glycidyl methacrylate monomers (GMA); d) irradiating with a 60-cobalt source and drying the obtained bi-structured matrix; e) incubating the matrix obtained in the previous step with a solution comprising iminodiacetic acid (IDA) and dimethyl sulfoxide (DMSO), and f) then incubating in the presence of an acid and drying.

The invention provides a process for preparing the matrix of claim 13, characterized in that the process comprises the following steps:

a) contacting a pipette tip as solid carrier with a suspension containing at least a hydrosoluble polymer and silica particles;

b) drying the pipette tip coated with the hydrosoluble polymer and silica, and immersing said pipette tip in an irradiation solution; and

c) irradiating with a 60-cobalt source and drying the obtained bi-structured matrix, where the silica particles have a diameter between 0.015 and 40 microns.

### DESCRIPTION OF THE FIGURES

FIG. 1: Coating degree (C.D. %) obtained in bi-structured matrices comprising rPUF coated with different formulations of hydrosoluble polymers, described in Table 1.

FIG. 2: Coating degree (C.D. %) obtained in bi-structured matrices comprising rPUF coated with 10%, 64 kDa PVA, with and without a coagulation treatment with 2-propanol previous to the drying process.

FIG. 3: Modification degree (G.D. %) obtained in the bi-structured matrices comprising rPUF coated with 10%, 64 kDa PVA, by irradiation in a solution with different amounts of GMA monomer.

FIG. 4: Modification degree (G.D. %) of bi-structured matrices comprising rPUF coated with 10% 64 kDa PVA, irradiated in the presence of a solution with different monomers.

FIG. 5: Scanning electron microscope images of a bi-structured matrix comprising original rPUF (A&C) and a bi-structured matrix obtained from rPUF and PVA (B&C) at different magnifications (200×, 2000×, and 10000×).

FIG. 6: FT-IR ATR spectra for bi-structured matrices comprising rPUF (a) with different hydrosoluble polymer coatings: (b) agarose, (c) PVA and (d) HEC. All samples were analyzed in dehydrated state.

FIG. 7: FT-IR ATR spectra performed on bi-structured matrices comprising: (a) rPUF coated with PVA: (b) rPUF coated with PVA and irradiated with GMA and (c) rPUF coated with PVA and irradiated with GMA and (d) derivatized with sodium sulfite. All samples were analyzed in dehydrated state.

FIG. 8: Amount of Copper²⁺ incorporated per gram of material as a function of the initial concentration of GMA monomer used for irradiating it.

FIG. 9: Elemental composition spectra (EDAX) obtained from retrodispersed electrons from SEM image (FIG. 5d) of the bi-structured matrix sample: rPUF-PVA-pGMA-IDA-Cu²⁺. Top graphic: internal zone of the material (image center); Bottom graphic: superficial zone of the material (image lower right quadrant).

FIG. 10: Langmuir's isotherm determination for Lysozyme adsorption by the bi-structured matrix comprising rPUF modified with sodium sulfite (rPUF-Sulfo). (* in equilibrium).

FIG. 11: Percentage of Green Fluorescent Protein (GFP-6×His) recovery from a protein extract by means of fluorescence determination, employing rPUF-PVA-pGMA-IDA-Cu²⁺ bi-structured matrix.

FIG. 12: Percentage of Fluorescein recovery (measured in relative fluorescence units (RFU)) for virgin micropipette tips (Tip) and for Tip-PVA and Tip-PVAcl bi-structured matrices, that had been previously charged with fluorescein and dried until constant weight.

FIG. 13: Percentage of Fluorescein recovery (measured in relative fluorescence units (RFU)) in sequential elutions of Tip-PVA and Tip-PVAcl bi-structured matrices, that had been previously charged with fluorescein and dried until constant weight.

FIG. 14: Pictures of bi-structured matrix DNA-Tips, obtained with silica microparticles of 40-63 microns (DGS) at 150 mg/mL, nanosilica of 0.020-0.040 microns in diameter (DNS) at 35 mg/mL and Fumed Silica of 0.015 microns in diameter (DFS) at 40 mg/mL.

FIG. 15: View with optical magnifying glass of different parts of a bi-structured matrix of the DNA-Tip kind prepared with DFS.

FIG. 16: Recovered DNA amount with a bi-structured matrix of the DNA-Tip DFS kind prepared at different pHs.

FIG. 17: Recovered DNA amount with a bi-structured matrix of the DNA-Tip DFS kind prepared at different concentrations.

FIG. 18: Agarose gel of DNA digestions with HindIII restriction enzyme at different times, with or without purification. Col: purification with a standard silica column; N.P.: non-purified sample; DNA Tip: purification with a bi-structured matrix of the DNA-Tip kind.

FIG. 19: Agarose gel of a RNA sample contaminated with RNAsa and subsequently purified with a bi-structured matrix of the DNA-Tip kind. All samples were incubated for 10 min at 37° C. before gel run. In all cases RNA presence is observed.

FIG. 20: Qualitative study on Premix-Tip efficiency. 1.5% Agarose gel of a DNA sample amplified with Premix-Tip and a DNA sample amplified with PCR standard premix. Lane 1: Molecular weight pattern; Lane 2: Reaction blank (Premix-Tip without template); Lane 3: PCR product using a Premix-Tip. Lane 4: PCR product using a standard mix.

FIG. 21: Qualitative study on bi-structured matrix of the Clean-Tip kind efficiency. 1.5% Agarose gel of a DNA sample non-treated with Clean-Tip and purified with Clean-Tip. Lanes 1 and 6: Molecular weight pattern; Lane 2: Reaction blank (standard PCR mix without template) without treatment with Clean-Tip; Lane 7: Reaction blank (standard PCR mix without template) with Clean-Tip treatment; Lanes 3 to 5: PCR product without Clean-Tip treatment; Lanes 8 to 10: PCR product with Clean-Tip treatment.

### DESCRIPTION OF THE INVENTION

The preparation of a bi-structured matrix comprising at least two different parts bound to each other, having a common surface, is shown. One part is a water-insoluble structure or polymeric solid carrier and the other part is a hydrosoluble polymer. The solid carrier provides physical rigidity by means a macroscopic structure and can have different formats, e.g. it can be a disposable micropipette tip or a reticulated porous material, open like a sponge. The hydrosoluble part provides water, and other solutes, absorption capability, and it comprises preferably polyvinyl alcohol (PVA), agarose or hydroxyethyl cellulose. The hydrosoluble polymer may further comprise other components like microparticles, nanoparticles or chemical molecules.

Materials are bound to each other by applying ionizing radiation application. Material binding occur when employing PVA, agarose or hydroxyethyl cellulose. Irradiation is performed in the presence of a certain amount of water, which facilitates cross-linking of materials.

The bi-structured matrix of the invention may be used for performing simple and fast laboratory assays, according to added functionalities, employing lower amount of containers and chemical reactants.

A new method for preparing a bi-structured matrix having a polymer structure or solid carrier and a hydrosoluble polymer coating which may have functional properties is disclosed. As raw materials, polymers of industrial application, readily available, are employed as raw materials.

The structure or solid carrier of the bi-structured matrix may have different shapes, e.g. flat sheets, tubes, container flasks, disposable micropipette tips or open-pore polymeric sponge (cross-linked). The minimum requirement therefor is to have a self-supporting structure, rigid enough as to keep the macroscopic shape of the final product. Cross-linked polyurethane sponges or foams (rPUF) are an industrial material, chemically inert, with three-dimensional structure, having excellent mechanical properties (high strength and elasticity), good availability and inexpensiveness. rPUFs have high porosity (about 97%) and a highly structured open macro-structure. Being an elastic material, the flexibility of rPUF foam also provides adequate stability and compression yield strength.

According to the method disclosed herein, it is obvious for a person skilled in the art that any solid carrier or structure can be employed to prepare the bi-structured matrix of the invention. For instance, the polymer solid structure or carrier may be a pipette tip, rPUF, container tubes of the Falcon® or Eppendorf® types or multi-well plates of the ELISA type.

The preparation of the bi-structured matrix of the invention is divided into different steps consisting of: (i) a physical coating of the surface of a polymeric solid carrier with a hydrosoluble polymer; (ii) cross-linking and setting thereof; and optionally specific functionalization with chemical ligands or particles in the hydrosoluble polymer. These two last steps can be performed simultaneously.

In a preferred embodiment the coating process is performed by immersion, usign rPUF as solid carrier.

For the coating process, three different hydrophilic polymer solutions can be used, e.g. a natural occurring polymer (agarose), a semi-synthetic polymer (hydroxyethyl cellulose—HEC) and finally a synthetic polymer (PVA).

Agarose is a polysaccharide forming a hydrogel at room temperature, with intrinsic neutral charge and highly hydrophilic.

HEC is a polymer soluble in water derived from cellulose. It is a non-ionic polymer which is compatible with a wide variety of other water soluble polymers. It is industrially used as thickener in latex paints and paper finishing.

PVA has excellent film forming properties with good flexibility, it is an inexpensive material and soluble in water. It is also used as thickener in many mass-use products. HEC and PVA coating polymers were tested in two different molecular weights.

Aqueous solutions were prepared for each one of hydrosoluble polymers above, at maximum possible concentration in the conditions detailed in Table 1.

TABLE 1
Polymer types used for the preparation of bi-structured
material. pH, work temperature and final achieved
concentrations for each one, are disclosed.

| Type | pH | Temperature ° C. | Concentration | |
|---|---|---|---|---|
| 64 kDA PVA | 7 | 85 | 5% | w/v |
| | | | 10% | w/v |
| 72 kDa PVA | 7 | 85 | 5% | w/v |
| | | | 10% | w/v |
| Agarose | 7 | 80 | 8% | w/v |
| 90 kDa HEC | 7 | Room temperature | 6.7 | w/v |
| 720 kDa HEC | 7 | Room temperature | 1.4 | w/v |

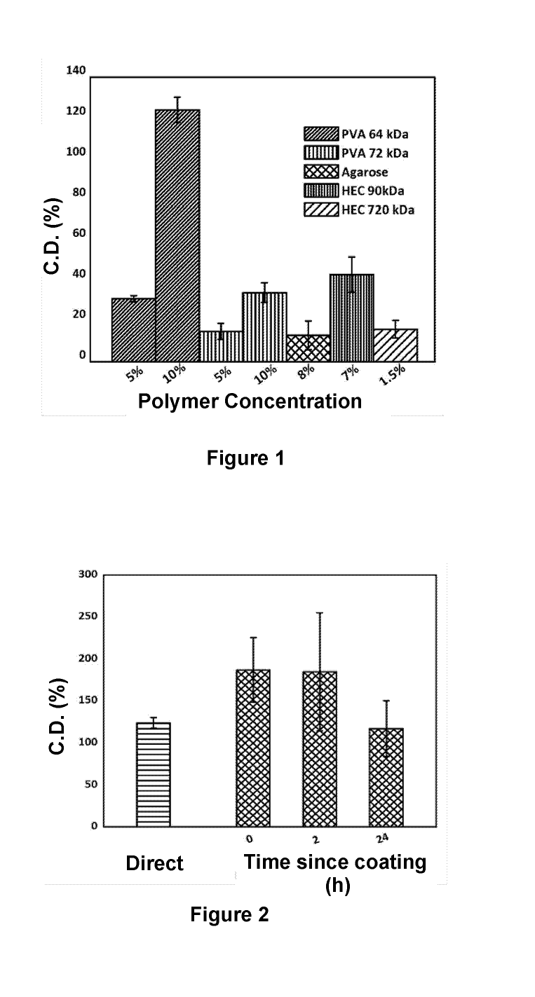

Ten rPUF were prepared, shaped like cylinders and were immersed into hydrophilic polymer solutions as described in Example 1. After drying the materials until constant weight the coating degree (CD %) was calculated. FIG. 1 shows the coating degree achieved for all prepared polymer types and concentrations. A greater coating polymer amount is assumed to provide a larger amount of hydrophilic layer in the bi-structured matrix.

The coating degree (CD %) was calculated as weight increase percentage:

CD %=100[W₁−W₀)/W₀],

where W₀ and W₁ are the initial weight and final (coated) weight of materials, respectively.

Agarose-based and high molecular weight HEC-based coating polymers have demonstrated a coating degree lower than 20%, similar to 72 kDa PVA at a low concentration. Low MW HEC showed a higher coating, more than twice the prior one, similar to 72 kDa PVA at a high concentration and 64 kDa PVA at 5%. The solution of 64 kDa PVA dissolved at 10% was the one providing the highest coating yield. For the purposes of the present invention, all the coating polymers turned out efficient as solid carrier coating.

It should be mentioned that the coating process was carried out at different temperatures, for example, at temperatures of about 20 to about 90° C., the highest temperatures allowing for reduced viscosity of the polymer solutions and improved the coating process.

In order to increase the coating degree and avoid leaching, a clotting step may be incorporated before the drying step. For clotting, 2-propanol may be used, which is for example, capable of clotting PVA. Other clotting agents may also be used, such as e.g., ethanol, 1-propanol or dioxane, all of which are within the scope of the present invention.

In case 2-propanol is used, samples are immersed in the solvent for ten seconds, then removed from the solvent and dried in a stove at 55° C. FIG. 2 illustrates CD % of the samples treated with 2-propanol for 10 seconds at different moments after the coating process and prior to the drying step. The coating process carried out previously was as follows: 64 kDa PVA 10%, pH 7 at 85° C. Each experimental condition corresponded to a group of ten cylinder-shaped rPUF.

According to the results shown in FIG. 2, the treatment with 2-propanol improves the CD %. Other coating conditions were studied, such as pH of the PVA solution. Four different pHs were tested (2; 5; 7; 7.5 and 9). All of the tested pHs allowed the formation of the coating. In a preferred embodiment the coating was carried out using a PVA solution at pH 7.5.

Another tested variable was the PVA solution's temperature. Coatings were performed at the following temperatures: 20° C., 40° C., 60° C. and 85° C. All temperatures allowed the formation of a suitable coating. In a preferred embodiment the temperature was 85° C.

The effect of the addition of a detergent to the PVA solution was assessed. Two PVA solutions were assessed having 2% of Triton X-100 and 2% of SDS. No improvement was observed in the CD % by modifying the moisturizing of the sample by the presence of the detergent.

In a preferred embodiment, the process comprised 64 kDA PVA 10%, pH 7.5, 85° C., and performing a clotting treatment with 2-propanol carried out before 2 hr after completing the immersion.

In order to improve hydrophilicity of the coatings, other hydrophilic substances may also be added, such as e.g., polyethylene glycols.

The bi-structured matrix comprising the solid carrier with the hydrosoluble polymer coating may be subject to a cross-linking and fixation step. For that, the matrix is immersed in a water-based solvent. The water-based solvent comprises a water and solvent ratio. The solvent may be ethanol or any other water compatible solvent producing a partial clotting of the polymer, maintaining said hydrosoluble polymer in close contact to the surface of the solid carrier. The presence of water generates a high amount of hydrated electrons and hydroxyl radicals during irradiation, due to water radiolysis. Radicals generated in the solvent react with the soluble polymer, e.g., PVA, and the surface of the solid carrier, thus generating macro-radicals. These macro-radicals may recombine with each other, generating new chemical bindings and thus, resulting in cross-linking (reticulation) of both matrix components.

The modification degree (GD %) was calculated as a weight increase percentage (Przybycien, 2004).

GD %=100[W₂−W₁)/W₁],

where W₁ and W₂ are the coated material weight and the final material weight, respectively.

In one embodiment, and as a way of example, the bi-structured matrix was immersed in an ethanol/water base solution, 1/1 (v/v) for the irradiation process, by air-tightly sealing the container. Dissolved oxygen in said solution was previously removed by bubbling nitrogen gas. The samples were irradiated with a 10 kGy dose at a dose rate of 1 kGy·h⁻¹. Irradiation was performed at room temperature using an irradiation source of 60-Cobalt (semi-industrial source PISI, CNEA, Ezeiza, Argentina).

The irradiation solution was prepared in different ways according to the experiment. After irradiation, the material was washed several times with water and ethanol 96% until all residues from the reaction were removed. The materials were dried for 24 hours in a stove at 55° C. until reaching constant weight.

The bi-structured matrix formed may be functionalized simultaneously if one or more monomers are added to the mixture to be irradiated. In this case, a polymerization reaction takes place induced by the irradiation process (RIIP). This polymerization reaction may occur on the hydrosoluble polymer, for example in the PVA, producing a modified polymer. With the purpose of increasing the yield of RIIP, the irradiation process needed to be carried out at a low dose rate.

The glycidyl metacrylate (GMA) monomer is widely used since it has a reactive epoxy group which allows several functionalization reactions in a simple way. Ionizing radiation, especially gamma radiation, ensures a high penetration into the sample, by which a modified material with a high homogeneity degree is obtained. Other monomers may be used, for instance, 2-hydroxyethyl metacrylate, metacrylic acid, dimethyl acrylamide (DMAAm) and metacrylic anhydride, all of which fall within the scope of the present invention.

For the purposes of the present application, when making reference to glycidyl metacrylate (GMA), dimethyl acrylamide (DMAAm), or other monomers, it is understood that said monomer is found in the form of a polymer produced by said monomers.

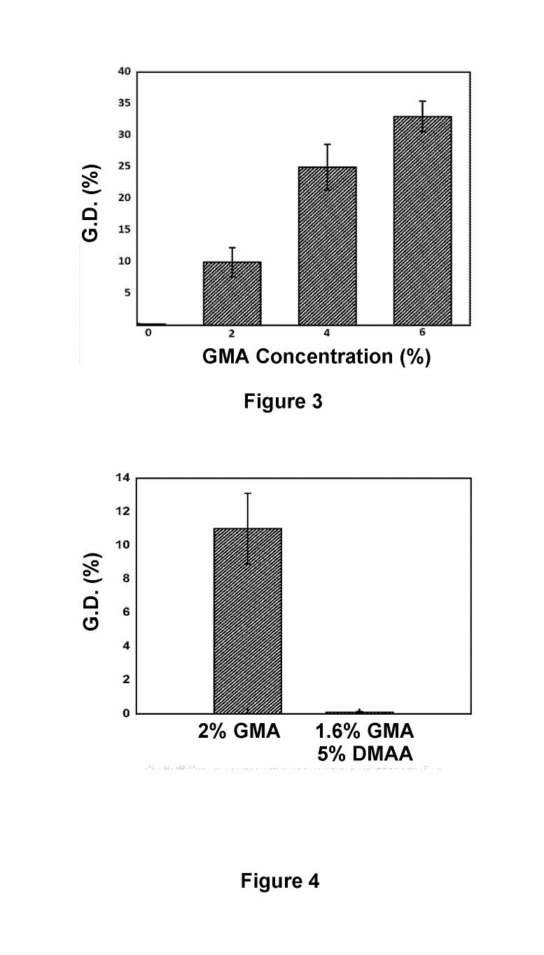

In order to remove the possible formation of homopolymers after the irradiation process, the material was washed. Then, it was dried and weighed for calculating of the modification degree percentage, as GD %, corresponding to the increase in percentage weight relative to the coated polymer. In FIG. 3 the amount of added (grafted) polymer is plotted as GD % as a function of the initial concentration of the GMA monomer in the irradiation solution. As expected, a direct relationship is found between these variables.

Then, the ratio between gained weight of PVA adsorbed in the coating and gained weight corresponding to the graft was calculated (Table 2), thus determining the final added polymer weight/PVA weight ratio. As may be seen, this ratio increases proportionally with the initial monomer content in the irradiated sample.

TABLE 2
GMA Monomer percentage used during the irradiation process
and modification degree (GD %) obtained and mass ratio of
the different polymers in the final bi-structured matrix

| | | Added polymer |
|---|---|---|
| [% GMA] | GD % (total) | weight/PVA weight |
| 2 | 10 | 1.1 |
| 4 | 25 | 1.25 |
| 6 | 33 | 1.33 |

In a following step, the addition of a second monomer, DMAAm, during the modification of the polymer was assessed, which increases hydrophilicity. The DMAAm monomer was incorporated into the polymer irradiation solution for obtaining co-polymerization with GMA.

FIG. 4 shows the results of the addition of DMAAm as a co-monomer. The addition of this second monomer did not result in the increase of modification degree of the material (GD %).

The presence of polyGMA (pGMA) in the functionalized bi-structured matrix allows for other chemical modifications and addition of new functionalities to said matrix. For example, sulfonic groups were added by incubation with a sodium sulfite solution (see Examples). Then, the residual epoxides were inactivated to diols in acid medium. Herein below, the protein adsorption ability of the sulfonic bi-structured matrix is shown.

Functionalization of the bi-structured matrix with iminodiacetic acid (IDA) group was performed by incubating in an IDA solution, as described in the examples. For the process of RIIP functionalization, acrylic, metacrylic and acrylamide monomers were used. For example, GMA and DMAAm were used as monomers. GMA provides a reactive epoxy ring and DMAAm provides hydrophilic properties.

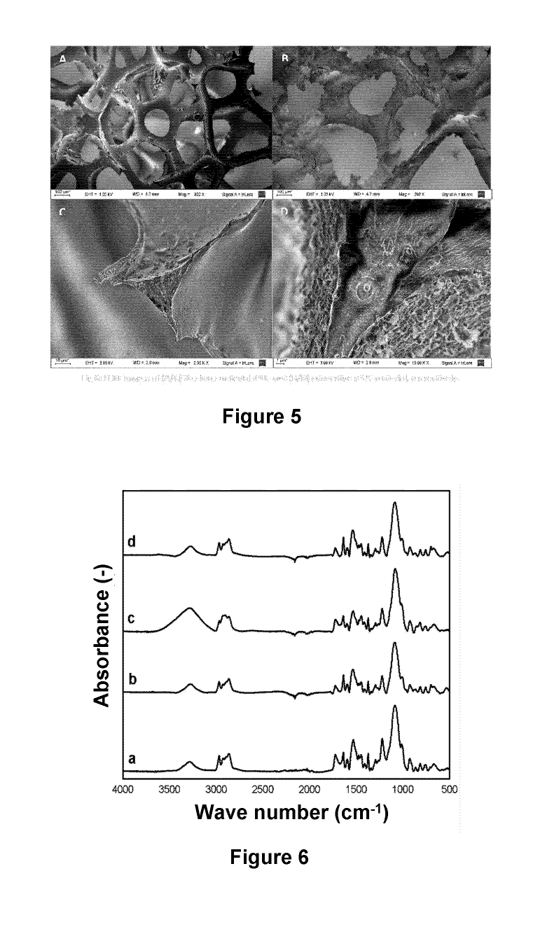

In FIG. 5 SEM images are shown of the original rPUF samples and of the bi-structured matrix of the invention at magnifications of 200×, 2000× and 10000×. FIGS. 5.b and 5.d the changes occurred on the surface of the material when compared to the original (FIGS. 5.a and 5.c), can be observed. It may also be observed that the bi-structured matrix keeps the physical (tridimensional) structure of the base material intact.

Chemical changes on the matrix were analyzed by infrared spectroscopy, more particularly, Fourier transform infrared spectroscopy with attenuated total reflectance (FTIR-ATR). Given the solid materials characteristics, which many times do not allow the infrared light to pass through, the attenuated total reflectance technique was used. This technique allows to analyze the surface layers of polymers.

The spectra corresponding to agarose (FIG. 6.b) and HEC (FIG. 6.d) coatings showed very slight changes with respect to the base material, rPUF (FIG. 6.a). The spectrum corresponding to the PVA coating (FIG. 6.c) showed a significant increase of signal at 3300 cm⁻¹ which correlates with hydroxyl groups and a signal close to 2900 cm⁻¹ corresponding to the stretching of CH₂ groups in PVA.

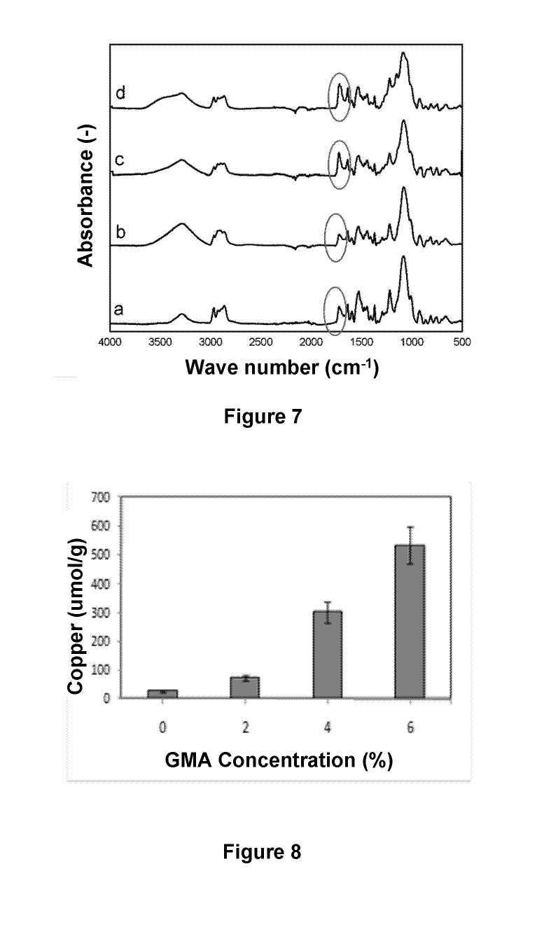

FIG. 7 shows the FTIR-ATR spectra corresponding to an original rPUF (FIG. 7.a), bi-structured matrix: rPUF with PVA coating (FIG. 7.b), rPUF bi-structured matrix with PVA coating and pGMA modification (FIG. 7.c), and with the sulfonic functionality (FIG. 7.d). In spectrum (b) of FIG. 7 the increase in signal at 3300 cm⁻¹ of the hydroxyls is observed again. In the spectrum of FIG. 7.c a proportional increase of the carbonyl signal (1720 cm⁻¹) is observed, but in this case, it is assigned to pGMA. In the spectrum of FIG. 7.d the characteristic peak at 1100-1000 cm⁻¹ is observed corresponding to the sulfonic groups. The signal at 3500 cm⁻¹ in this spectrum may be due to water residues in the sample (it rapidly hydrates).

It is important to note that in the ATR technique the degree of penetration of the infrared waves is not the same for different frequencies and it has a complex dependency with the refraction index and the type of crystal used to place the sample. Therefore, the FTIR-ATR spectra are only used for detecting the typical bands, especially in the shorter wavelength regions.

With the aim of obtaining additional information on the presence of functionality within the hydrophilic region of the bi-structured matrix of the invention comprising the modification with pGMA, an iminodiacetic acid (IDA) immovilization was carried out as described in the examples. IDA is capable of reversibly chelating metallic ions such as Cupper 2+. This property allowed for the quantification of the amount of available ligands by quantifying of ion eluted with a higher-affinity chelating agent such as EDTA. For estimating the incorporated micromoles, a calibration curve was built using Cupper 2+, the R² of which was 0.9979. Absorbance data were interpolated in the curve, then referred to the weight of analyzed material (FIG. 8). This assay allowed to prove without beyond doubt that the materials had effectively been modified with pGMA. The amount of ligand was also proportional to the amount of pGMA in the sample. It is evident that IDA, ethylene diamine (EDA), 2-mercaptoethanol and sulfite may be used for modifying the material.

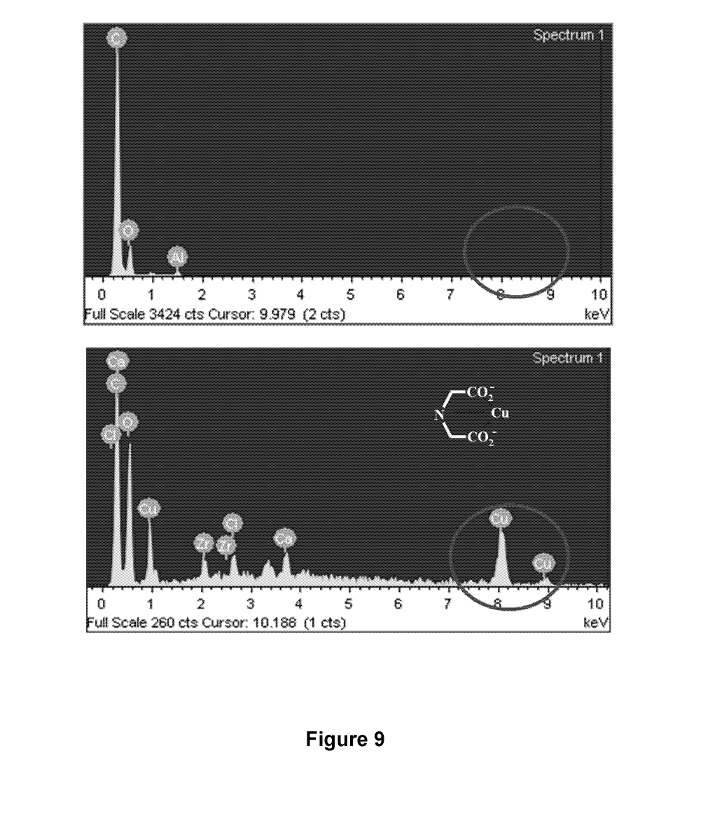

In order to confirm whether the monomer is bound to the hydrophilic polymer, an elemental analysis was performed on the scanning electron microscopy images (SEM-EDAX). FIG. 9 shows the element analysis spectra as obtained in the interior of the rPUF material (obtained on the central part of the image of FIG. 5.d) and the spectrum corresponding to the same image on the surface zone thereof (bottom right quadrant) corresponding to a sample of the bi-structured matrix functionalized with IDA-Cupper²⁺. It may clearly be observed the presence of cupper on the matrix surface but not in the interior, thus indicating the binding of the functional monomer+IDA-Cupper²⁺ to the hydrophilic polymer.

The sulfonic bi-structured matrix is useful for protein purification. An interior coating was prepared according to the methodology shown in the examples, on top of an open-pore polyurethane foam (rPUF) cylinder. The 64 kDa PVA solution was used. 4% GMA was added to the irradiation solution. Next, the bi-structured matrix comprising rPUF thus modified was treated with the sulfite solution as described in the examples for finally obtaining the sulfonic bi-structured matrix (rPUF-Sulfo).

The rPUF-Sulfo matrices were equilibrated in phosphate buffer pH 7 and incubated with a Lysozyme solution. The protein was reversibly adsorbed on the material, and then it was possible to completely elute the same using a high ionic strength solution, for example, 1M NaCl.

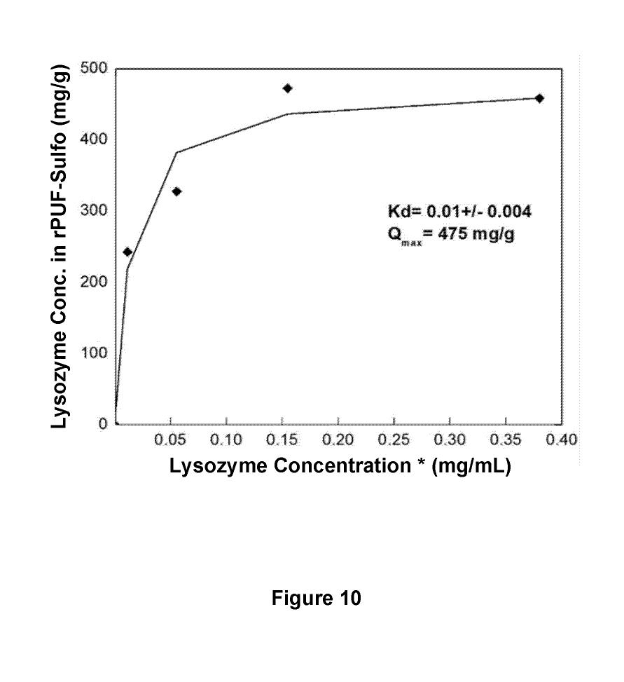

Maximum saturation capacity was determined by incubating the rPUF-Sulfo with variable enzyme amounts. In this assay, through analysis of the Langmuir's isotherm, the maximum saturation capacity (Qmax) and the dissociation constant (Kd) were determined by non-linear fitting of the experimental data (see FIG. 10). FIG. 10 shows that the PUF-Sulfo adsorption behavior corresponds to a protein ion exchange chromatographic matrix.

The bi-structured matrix comprising immobilized IDA-Cupper²⁺ for purification of histidine-tagged proteins was used. An inner coating was prepared according to the methodology developed in the examples on top of an open-pore polyurethane foam (rPUF) cylinder. The 64 kDa PVA solution was used. An amount of 2%, 4% or 6% of GMA was added to the irradiation solution and then, the rPUF was treated with the IDA and Cupper²⁺ solution as described in the examples, in order to obtain the IDA-Cupper²⁺ bi-structured matrix (rPUF-PVA-pGMA-IDA-Cu²⁺).

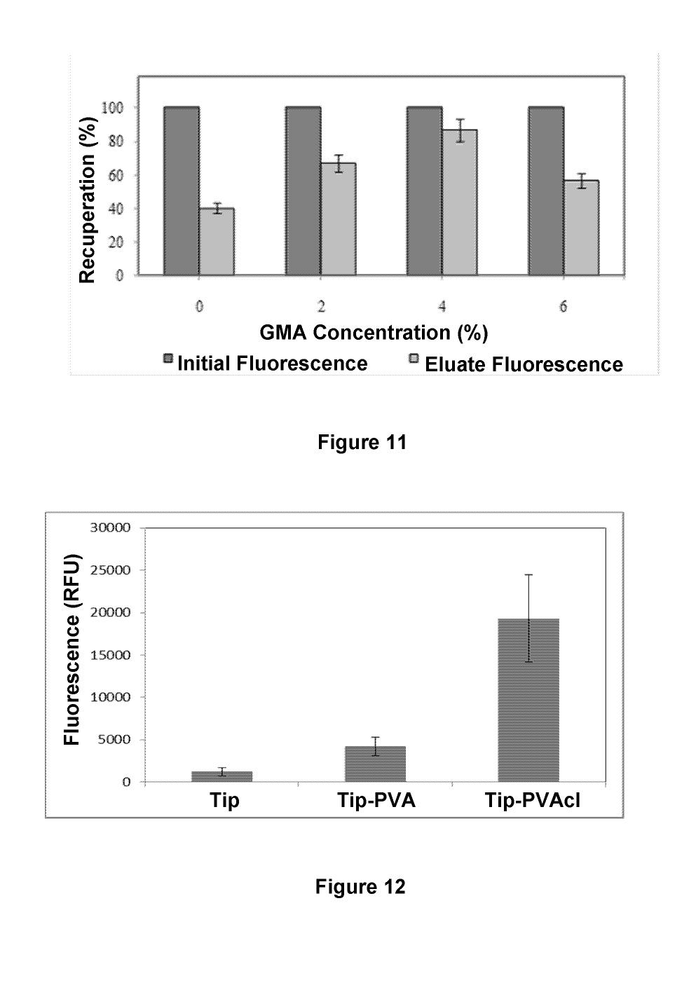

Then, an homogenate of a recombinant E. coli strain was obtained expressing the Green Fluorescent Protein with a terminal 6-histidine sequence (GFP-6×His). The biomass was harvested and homogenized with a tip sonicator. The cell lysis product was then subjected to Molecular Exclusion Chromatography with a pre-packed PD10 column containing Sephadex® G-25 M for changing the buffer solution at pH 7 and removing the medium where the protein was contained. The eluted macromolecules fraction was used for testing the specific adsorptive capacity of the rPUF-PVA-pGMA-IDA-Cu²⁺ matrix of the invention. The GFP-6×His content in the samples was determined by fluorescence in a Nanodrop 3300 apparatus. FIG. 11 shows the initial fluorescence of the homogenate and the fluorescence after purifying GFP-6×His by a process involving incubation of the homogenate with the rPUF-PVA-pGMA-IDA-Cu²⁺ bi-structured matrix, washing with buffer solution and eluting with imidazole solution. FIG. 11 shows that it is possible to efficiently recover the protein by using any of the bi-structured matrices used, under different conditions. In a preferred embodiment, a rPUF-PVA-pGMA-IDA-Cu²⁺ bi-structured matrix generated from an irradiation solution with 4% GMA was used.

In another preferred embodiment, the bi-structured matrix comprises a polymeric solid carrier consisting of a micropipette tip and a hydrophilic polymer, for example, PVA, among others. For this, an inner coating was prepared according to the methodology described in the examples for a 300 μl disposable virgin micropipette tip (Tip) using PVA. Tips were prepared with the PVA solution coating without the irradiation process (Tip-PVA) and applying the irradiation cross-linking process (coating and reticulation), thus referring to this matrix as Tip-PVAcl.

The different materials (Tip/Tip-PVA/Tip-PVAcl) were incubated with 200 μl of a fluorescein solution, for 1 minute. Next, the content was discarded without leaving drops inside. The materials were dried in a stove for 15 minutes at 60° C. This procedure was repeated twice. Then, the loaded Tips were left to dry in a stove at 40° C. overnight.

The fluorescein-loaded Tips were placed into a p200 micropipette, graduated at 200 μl. Also, 50 μl of 50 mM phosphate buffer solution were loaded into Eppendorf® tubes. Then, the buffer solution from the tubes was allowed to run over the inner wall of the fluorescein-loaded Tips. The process was repeated five times for each type of Tip (samples by triplicate). Finally, the fluorescence of the solution eluted in each tube was determined in a NanoDrop 3300 fluorospectrometer.

FIG. 12 shows the total amount of fluorescein (measured as relative fluorescence units—RFU) eluting for each type of Tip. The Tip-PVAcl clearly shows an amount of fluorescein which is ten times higher than a virgin Tip and 5 times higher than a Tip-PVA.

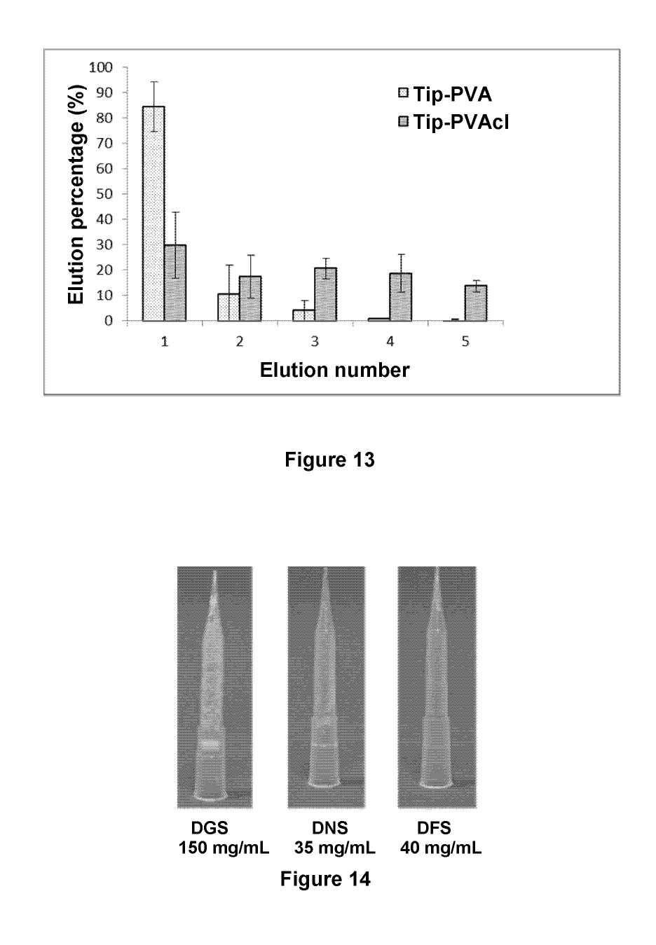

Additionally, the reactant (Fluorescein) discharge process was different, depending on whether the PVA was physically immobilized (Tip-PVA) or immobilized and reticulated by irradiation (Tip-PVAcl). FIG. 13 shows the relative percentage elution of the fluorescein content from Tip-PVA and Tip-PVAcl in the sequential elutions. The Tip-PVA releases more than 80% of the product in the first elution and almost 100% in the first two elutions. On the contrary, the Tip-PVAcl releases a constant amount of product during the first four elutions. Accordingly, different modifications may be used for different applications depending on the operator's needs. It is important to point out that, along with the product, the Tip-PVA will release part of the PVA used for the coating.

This reactant loading procedure in the Tip-type bi-structured matrices is possible with different organic molecules, and it is also possible to keep it dehydrated for a long time before its final use.

Use of the micropipette tip-type bi-structured matrix for nucleic acid analysis: a 300 μl Tip-PVAcl was prepared according to the examples. A DNA quantification reagent (PicoGreen®), which generates fluorescence only with the presence thereof, was loaded. For this, a Tip-PVAcl with 200 μl of the Pico Green® reagent was incubated in a 1/200 dilution of the commercial stock (PicoGreen® Reactive) for five minutes. The content was discarded without leaving drops inside. The Tip-PVAcl were dried in a stove for 15 minutes at 60° C. These steps were repeated twice. Then, they were left to dry in a stove at 40° C. overnight. The Tips loaded with PicoGreen® are hereinafter referred to as Quanti-Tip and constitute an embodiment of the matrix of the invention.

Then, the reagents discharge was carried out (Application—DNA quantification). For this, a plasmidic DNA extraction was performed according to the standard preparation procedure known as MINIPREP. Dilutions of a plasmidic DNA extraction (1/10, 1/100 and 1/500) were prepared in water. 20 μl of each dilution were placed into Eppendorf® tubes 0.5 ml in volume. A Quanti-TIP matrix was placed in a micropipette and calibrated to a volume of 200 μl. The plunge was completely pulled back and the 20 μl of the DNA sample present in the tube were collected. The micropipette plunge was moved up and down so that the content would slowly move over the interior of the Quanti-Tip about 6 times. Finally, the Quanti-Tip matrix′ content was eluted in another sterile Eppendorf® tube. The eluate fluorescence is measured in a NanoDrop 3300. The obtained fluorescence value corresponds to a DNA concentration of 5 μg/mL for the highest dilution of the sample. This way, quantification of the amount of DNA present is accomplished by releasing the PicoGreen® dye from the Quanti-Tip matrix, which reacts with the DNA of the sample.

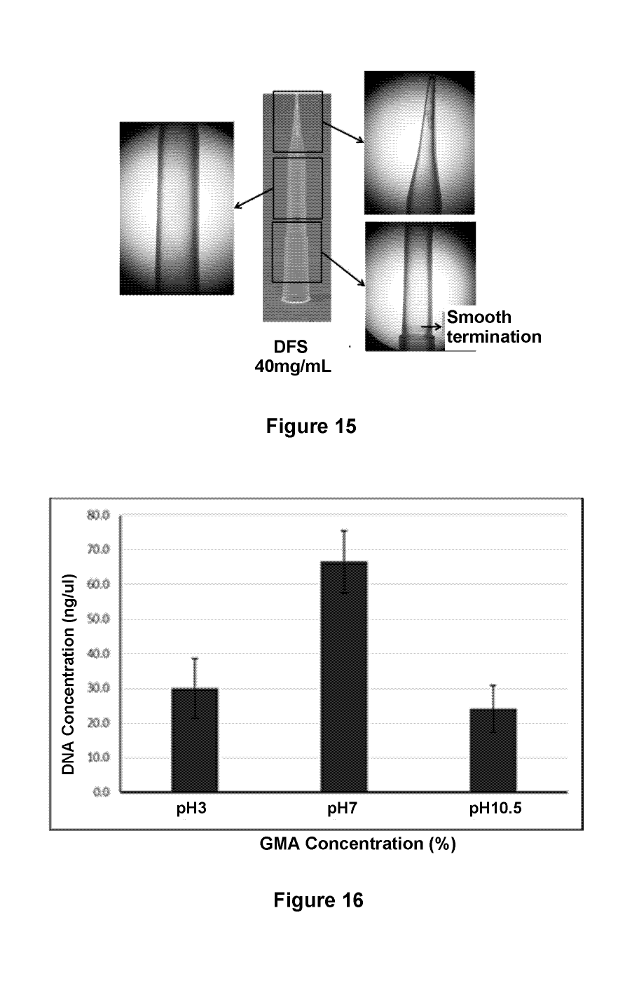

Use of the disposable micropipette tip-type matrix for purification of nucleic acids (DNA-Tip): an inner coating was prepared according to the methodology shown in the examples for a 300 μl disposable micropipette tip (Tip) using PVA. To the PVA solution used for the coating, during the material's preparation step, a silica particles solution was added. Micro-scale particles were used (40-63 microns (DGS)), and in the nanometric scale, with nanoparticulated silica, Nanosilica 0.020-0.040 microns in diameter (DNS) and Fumed Silica 0.015 microns in average particle diameter (DFS). The following concentration ranges were used: DGS 50 to 150 mg/mL and the DFS and DNS nanoparticles, between 10 and 50 mg/mL. FIGS. 14 and 15 show the DNA-Tip bi-structured matrices obtained with the different materials, showing the magnification of different sections of the DNA-Tip (DFS) with an optical magnifying glass. The nanoparticle-containing bi-structured matrices turned out to be more homogeneous and stable in time, thus achieving a more reproducible manufacturing technique (FIG. 15).

The different micropipette tips of the invention were analyzed according to the protocol described in the examples. Table 3 below shows the results for the DNA purification using the three tips.

TABLE 3
Amount of DNA recovered and quality analysis
according to coefficient 260/280

| | Eluted DNA | Standard | |
|---|---|---|---|
| DNA-Tip | (ng/μl) | deviation | 260/280 nm |
| sample | (average) | (ng/μl) | ratio |
| DSG | 35.5 | 8.5 | 1.9 |
| DNS | 40.8 | 2.7 | 2.1 |
| DFS | 80.8 | 18.5 | 2.0 |

Assays Carried Out in Triplicate

The DNA adsorption capacity using the bi-structured matrix containing DFS (DNA-Tip DFS) was twice the adsorption capacity obtained with other matrices. Additionally, the 260/280 ratio close to 1.8 indicates a protein-free sample. The bi-structured matrix containing DFS corresponds to the best appearance, as may be seen in FIG. 15.

Then, the effect of the pH in the process for obtaining the bi-structured matrix containing DFS was assessed. The matrix prepared at a neutral pH showed the best optical characteristics, stability over time and homogeneity. However, all the used pH values were suitable. The adsorption capacity of the DNA-Tip (DFS) matrices was also assessed at different pH values (FIG. 16). FIG. 16 shows that a neutral pH solution has a higher yield in the recovery of DNA.

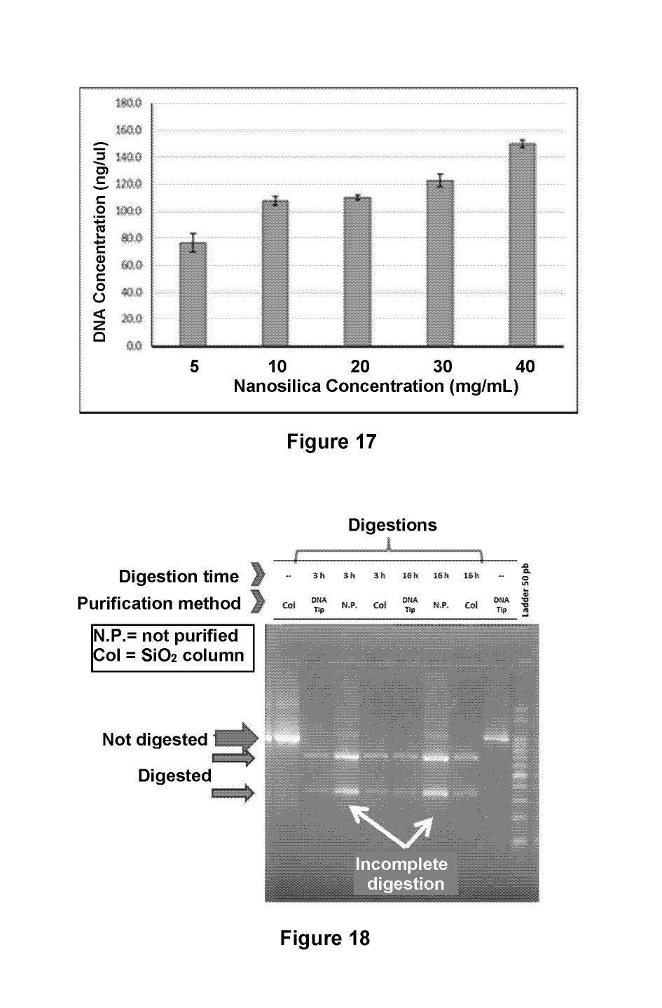

Lastly, the nanoparticulated silica concentration was analyzed. FIG. 17 shows the ratio of adsorption to amount of immobilized adsorbent (Fumed silica). A higher concentration lead to a higher recovery capacity, up to the maximum nanosilica concentration which allows the coating to be made (40 mg/mL).

Use of the DNA-Tip type bi-structured matrix for the purification of DNA: 300 μl disposable micropipette tips were prepared with the PVA coating and the addition of silica nanoparticles as described in the examples. This bi-structured matrix was called DNA-Tip. A nucleic acid purification protocol was performed on a pure DNA sample (a PCR-amplified 500 bp fragment) and on a E. coli DNA sample prepared as described in the examples.

4 μg of a PCR-amplified DNA fragment were purified with a DNA-Tip. 15 replicates were performed and the obtained samples were analyzed spectrophotometrically (Nanodrop 1000), by fluorometry (Qbit—Invitrogen) and by agarose gel electrophoresis. A purification system by means of silica columns was used as a reference methodology (Clean up—Productos Bio-Lógicos SA).

Table 4 below shows the data from the application of DNA-Tips. The assay was carried out on 15 identical samples in order to produce statistical data. The DNA recovery capacity with the DNA-Tip-type bi-structured matrix was 100 ng, with a reproducibility close to 80% and good quality parameters. This concentration is at the detection limit of the agarose gel electrophoresis.

The quality of the purified DNA was also assessed. The UV-vis spectrophotometry was used as the DNA quality method of analysis. In order to evaluate the quality of the purified DNA, the absorbance at 280 nm, 260 nm, 230 nm was assessed, considering the absorbance ratio 260 nm/280 nm>1.8 as a purity index with respect to protein contamination, and the absorbance ratio 260 nm/230 nm>2.0 as a purity index with respect to hydrocarbons, phenols, aromatic compounds and peptides.

TABLE 4
Capacity and quality parameters in DNA purification

| | | | | | Mass | Quality |
|---|---|---|---|---|---|---|
| | | Max. | | | repro- | repro- |
| | Replicate | capacity | Quality | Quality | ducibility | ducibility |
| | number | (ng) | 260/280 | 260/230 | % | % |
| DNA- | 15 | 100 ± | 1.83 ± | 2.3 ± | 78 | 95 |
| Tips | | 10 | 0.07 | 0.3 | | |

Reproducibility = [1 − (SD/average)]*100

The quality of the purified DNA was evaluated. Molecular biology methodologies require reagents and materials of a high purity in order to work properly. Generally, samples with presence of proteins (DNAse, RNAse, or others), oligonucleotides or trace of chemical reagents (phenol, GuCl, salts) may affect the efficiency of the techniques. One of the most sensitive methodologies in terms of the presence of proteins, oligos and other contaminants is sequencing. For this methodology to be efficient and to allow for a correct and complete reading of the bases of a DNA fragment (<1000 bp) the sample must be ultrapurified. The purification products from the DNA-Tip-type matrices were sequenced using a capillary system which allows the reading of up to 900 bases. As an additional assay, a DNA sample was deliberately contaminated with 16 μg of bovine serum albumin (BSA) prior to purification with the DNA-Tip-type bi-structured matrix.

TABLE 5
Quality parameters of a BSA-contaminated DNA sample

| | Sample | Purified sample |
|---|---|---|
| Mass (ng) | 4348 | 69 |
| Abs. (260/280) | 1.11 | 1.87 |

As may be seen on Table 5 above, the contaminated sample is correctly purified with the DNA-Tip-type bi-structured matrices, wherein the absence of BSA protein is shown. Then, all the samples were sequenced. When alignment of the purified fragments' sequences was carried out no differences with the template sequence were detected. All the samples purified with the DNA-Tip-type bi-structured matrix were sequenced with the same efficiency with respect to the control sample (silica columns), even the sample previously contaminated with BSA. Taking these results into consideration, it is deduced that the purified samples do not contain contaminants concentration levels that would affect the efficiency of a sequencing reaction, and hence, purification using the DNA-Tip-type bi-structured matrix is excellent.

In order to evaluate the performance of the purified products in other molecular biology methodologies, the same were subject to enzyme digestion reactions with the restriction enzyme HindIII. 8 purifications were carried out with the DNA-Tips and they were pooled into a single sample. The sample was concentrated by SpeedVac. FIG. 18 shows the purification of a DNA sample using the DNA-Tip matrix and a standard silica column. Enzymatic digestion was performed at two different times in order to ensure completion of the reaction. When controlling the samples without purification the reaction is incomplete both at 3 h and at 16 h of incubation (i.e. the band corresponding to the non-digested fragment is observed). Both in the sample purified with the DNA-Tip matrix and in the silica column, complete digestion is observed at both times. This result indicates that the amount of contaminants present (proteins, dNTPs, salts) is below the levels that would inhibit the enzymatic reaction (complete digestion).

Purification of RNA of the RNAse A Enzyme

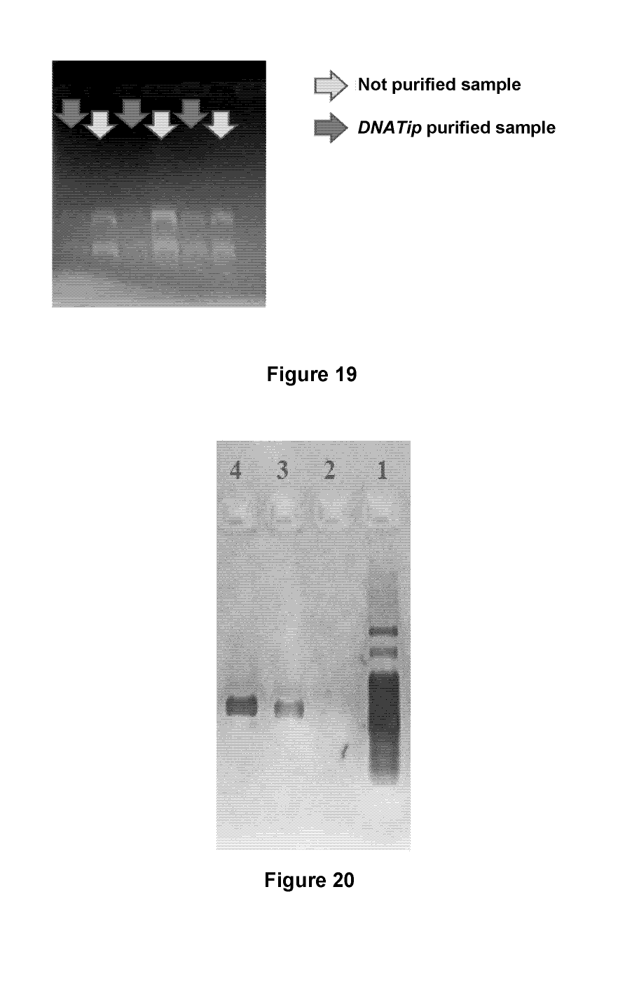

An eukaryote RNA sample was contaminated with the enzyme RNAse A and then purified with the DNA-Tip-type bi-structured matrix. Then, the obtained RNA was incubated for 10 min at 37° C. and the presence of RNA with respect to the original (non-contaminated) sample was visualized in agarose gels. Two remarkable results were observed in this assay: (i) the purification method with the DNA-Tip matrix is capable of purifying RNA by RNAse removal, and (ii) the DNA-Tip matrix holds RNA with the same or higher efficiency than for DNA (FIG. 19).

Use of the disposable micropipette tip bi-structured matrix for adding PCR reagents (Premix-Tip).

Reagent load (Premix for PCR): the Tip-PVAcl bi-structured matrix was prepared according to the examples. The Tip-PVAcl were incubated with 200 μl of qPCR Master Mix for 1 minute. The content was discarded without leaving drops inside. The materials were dried in a stove for 15 minutes at 50° C. These steps were repeated twice. Then, they were left to dry in a stove at 40° C. overnight, thus obtaining the Premix-Tip.

Reagent discharge (PCR Application). The PCR reaction mixture was made using a 20 μl primers solution and the fragment to be amplified with the Premix-Tip. The reagent discharge treatment with the Premix-Tip was as described for the TIP-PVAcl matrix loaded with fluorescein. Then, a standard PCR reaction cycle was performed. Finally, an aliquot of the amplification was taken and an agarose gel was run in order to corroborate amplification efficiency (see FIG. 20). FIG. 20 shows the use of the Premix-Tip yields an amplified DNA fragment similar to what is obtained with the use of the commercial Premix mixture.

Use of the Tip-PVAcl bi-structured matrix for purifying PCR reaction products.

An inner coating was prepared according to the methodology shown in the examples on a disposable micropipette tip, using a PVA solution to which 5 μm RP C-18 silica (Sigma) is added to a concentration of 20 mg/mL in a preparation process similar to the DNA-Tip bi-structured matrix. The Tip containing this matrix is hereinafter referred to as Clean-Tip

Purification of PCR Fragments (Application)

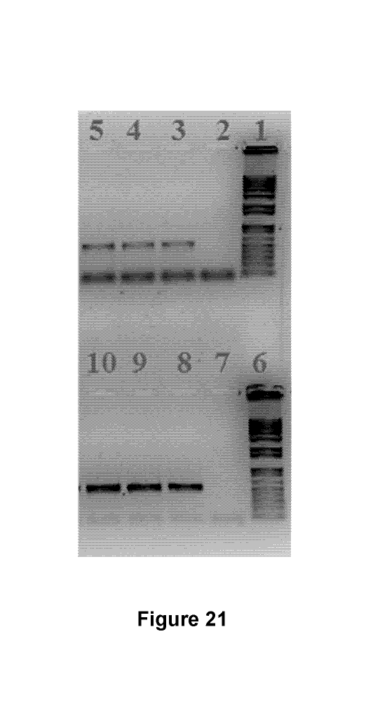

A PCR reaction was performed on a known fragment and in order to improve the quality of the amplified fragments, they were purified with the Clean-Tip bi-structured matrix. The integrity of the fragments was shown by comparing with the results of the same sample in an agarose gel (see FIG. 21). The lanes corresponding to the treatments with Clean-Tip show a lower content of contaminants (dNTP) in the sample.

The use of the Clean-Tip after the PCR reaction avoids the need of seeding all of the amplification product in an agarose gel for later recovery of the fragment from a pad thereof, a process that negatively impacts on the amount of DNA recovered.

The present invention is best illustrated by way of the following examples, which are not to be construed as a limitation on the scope thereof. On the contrary, it should be clearly understood that other embodiments, modifications and equivalents thereof may be practiced upon reading the present specification, all of which may be suggested to those skilled in the art, without departing from the spirit of the present invention and/or the scope of the annexed claims.

### EXAMPLES

Materials

Polyetherurethane and polyesterurethane (rPUF) open pore foam (cross-linked) with a pore size of about 250 microns were obtained from Eurofoam Deutschland GmbH. Product code: Filtren™ 60. The material was cut into cylinders of 0.4 cm diameter, 2 cm high (approximately 0.01 g weight).

Disposable polyethylene micropipette tips having 300 μl capacity (DIAMOND D300) were purchased from Bio-ESANCO, trade name GILSON. Eppendorf-type polyethylene tubes 1.5 and 2 mL in volume were purchased from the local market.

64 kDa and 72 kDa polyvinyl alcohol (PVA), 90 kDa hydroxyethylcellulose (HEC), 720 kDa hydroxyethylcellulose, glycidyl metacrylate (GMA), dimethyl acrylamide (DMAAm), Fluorescein, Fumed Silica and RP C-18 Silica (reverse phase) were obtained from Sigma-Aldrich Argentina. D1-Max agarose was obtained from Biodinamica SRL. Quant-iT™ PicoGreen® was purchased from Invitrogen Argentina. PCR Premix, acetone, anhydrous sodium sulfite, isopropanol, ethanol and lysozyme were purchased from the local market. All the other chemical products used were analytical grade.

### Example 1: Preparation of the Bi-Structured Matrix Using rPUF Foam as the Solid Carrier

Ten rPUF are cut with a cylinder shape. They are immersed for 10 seconds in a 64 kDa PVA 10% solution at 85° C. avoiding bubbles to be left inside. This was also carried out using agarose and hydroxyethylcellulose. Then, the material is drenched in order to remove soluble polymer in excess. Within the two following hours the pieces are immersed in 2-propanol for ten seconds. The pieces of material are dried for 24 hr in a stove at 55° C. until constant weight.

The dried pieces are immersed in 20 mL of irradiation solution made of ethanol/water (1/1 v/v) in a glass jar. Gas nitrogen is bubbled for removing dissolved oxygen from the solution. The jar is air-tightly closed. The jar is irradiated with a source of 60-Cobalt with 10 kGy at a dose rate of 1 kGy/h. After irradiation, the material is washed with water and 96% ethanol sequentially three times and dried in a stove at 55° C. until constant weight.

### Example 2: Preparation of the Bi-Structured Matrix Functionalized with Monomers as the Chemical Ligands

Ten rPUF are prepared having a cylinder shape and coated with PVA or other polymer as described in Example 1.

Irradiation is carried out as described in Example 1, except for the addition of 2, 4 and 6% GMA to the irradiation solution. The DMAAm monomer was also used, either alone or combined with GMA.

### Example 3: Preparation of the Sulfonic Bi-Structured Matrix (rPUF-Sulfo)

Ten rPUF having a cylinder shape are prepared according to Example 2. The dried material is incubated in 20 mL of a sodium sulfite/isopropanol/water (10/15/75 w/w/w) solution at 37° C. overnight. Then, the material is incubated in a 0.5 M H₂SO₄ solution at 80° C. for 2 hr. Then, the material is washed with abundant water and finally with 96% ethanol. The material is dried in a stove at 55° C. until constant weight.

### Example 4: Preparation of the Bi-Structured Matrix Comprising Iminodiacetic Acid (IDA) and IDA+Cupper2+

Ten rPUF having a cylinder shape are prepared according to Example 2. The dried material is incubated in 20 mL of a, for example, IDA solution (1M pH:11):DMSO (1:1) overnight, at 80° C. Then, the material is incubated in a 0.5 M H₂SO₄ solution at 80° C. for 2 hr. Then, the material is washed with abundant water and finally with 96% ethanol. The material is dried in a stove at 55° C. until constant weight. Ethylenediamine (EDA), 2-mercapto ethanol were also used instead of IDA.

The dried material is incubated with a 5% w/v solution of CuSO₄ with stirring (100 rpm) for 1 hr. It is washed with abundant water. Then, the cylinders are incubated with an 0.1 M EDTA solution at pH 7, for 1 hr, with mild stirring. The Cupper²⁺ content is determined in the eluted solution by determining the concentration of the EDTA-Cupper²⁺ complex by UV-vis spectrophotometry at 715 nm.

In the case of using IDA, the bi-structured matrix of the invention is called rPUF-PVA-pGMA-IDA-Cu²⁺

### Example 5: Preparation of the Bi-Structured Matrix Comprising a Solid Polymeric Support Consisting in a Micropipette Tip (Tip-PVA and Tip-PVAcl)

10 mL of a 72 kDa PVA 10% solution are prepared under stirring at 85° C. After complete dissolution of PVA the solution is kept at 40° C. A Tip is inserted in a P200 micropipette graduated to its maximum capacity (200 μl). Next, the PVA solution is slowly loaded in the interior of the Tip, it is kept loaded and in a vertical position for 30 seconds and finally, the content is discharged. The micropipette Tip is released and left to dry in a stove at 55° C. until constant weight. This material is called Tip-PVA.

The dried pieces (Tip-PVA) are placed in a glass jar and immersed in 20 mL of irradiation solution comprising ethanol/water (1/1 v/v). Gas nitrogen is bubbled for removing dissolved oxygen from the solution. The jar is air-tightly closed. The jar is irradiated with a source of 60-Cobalt with 10 kGy at a dose rate of 1 kGy/h. After irradiation, the material is washed with water once and finally with ethanol 96%. Then, it is dried in a stove at 55° C. until constant weight. This material is called Tip-PVAcl.

### Example 6: Preparation of the Bi-Structured Matrix of the Tip-PVAcl Type, Charged with Fluorescein

A 300 μl Tip-PVAcl is prepared according to Example 5. The Tip is placed in a P200 micropipette and loaded with 200 μl of a 40 μM fluorescein solution for one minute. The contents are discarded without leaving drops inside. The Tip is dried in a stove for 15 minutes at 60° C. This step is repeated twice. Then, it is left to dry in a stove at 40° C. overnight.

### Example 7: Preparation of the Bi-Structured Matrix of the Quanti-TIP Type

A 300 μl Tip-PVAcl is prepared according to Example 5. The Tip is placed in a P200 micropipette and is loaded with 200 μl of Pico Green reagent, diluted 1/200 from commercial stock (Quant-iT™ PicoGreen) for five minutes. The content is discarded without leaving drops in the interior thereof. The Tip is dried in a stove for 15 minutes at 60° C. These steps are repeated twice. Then, it is left to dry in a stove at 40° C. overnight. The PicoGreen-loaded Tip with is called Quanti-Tip.

### Example 8: Preparation of the Bi-Structured Matrix of the DNA-Tip Type, which Comprises Silica Particles

10 mL of a 64 kDA PVA 10% solution were prepared under stirring and at 85° C. after complete dissolution of PVA, Fumed Silica is added at a final concentration of 40 mg/mL. The mixture is kept under stirring and at 40° C. A Tip is inserted in a P200 micropipette and the PVA/silica suspension is slowly loaded inside. The solution is kept for 30 seconds and then the content is discharged. The micropipette Tip is released and left to dry in a stove at 55° C. until constant weight.

The dried pieces are immersed in 20 mL of irradiation solution comprising ethanol/water (1/1 v/v) in a glass jar. Gas nitrogen is bubbled for removing dissolved oxygen from the solution. The jar is air-tightly closed. The jar is irradiated with a source of 60-Cobalt with 10 kGy at a dose rate of 1 kGy/h. After irradiation, the material is washed with water and ethanol 96% and dried in a stove at 55° C. until constant weight. This material is called DNA-Tip.

This example was also carried out using micro scale particles, of 40-63 microns (DGS), and in the nanometric scale, with nanoparticulate silica, Nanosilica having a diameter of 0.020-0.040 microns (DNS) and Fumed Silica having an average particle diameter of 0.015 microns (DFS). The following concentration ranges were used: DGS from 50 to 150 mg/mL and the DFS and DNS nanoparticles, between 10 and 50 mg/mL.

### Example 9: Characterization of the Bi-Structured Matrix

Scanning Electron Microscopy (SEM)

SEM images were obtained from a SEM-Carl Zeiss NTS-SUPRA 40 microscope at a voltage of 3 kV. All samples were equilibrated in 3M KCl in phosphate buffer and rinsed with distilled water. Samples were dried in a stove at 55° C. until constant weight and examined at different magnifications. By means of an element analysis probe, the composition of the different structures of the sample was determined.

Fourier Transform Infrared Spectroscopy with Attenuated Total Reflectance (FTIR-ATR)

FTIR-ATR spectra were run on dried samples by directly measuring in a FTIR IRAffinity (Shimatzu Corporation) apparatus, equipped with an attenuated total reflectance GladiATR accessory (Pike Technologies, USA) with a single-reflection diamond crystal. The spectra were acquired by means of the average of 32 scannings in the wave number interval of 500 to 4000 cm⁻¹ with a resolution of 4 cm⁻¹ and analyzed with IRsolution Shimatzu 1.50 software.

Determination of the Protein Adsorption Capacity.

The static adsorption capacity of functional rPUF was determined in order to determine the Langmuir isotherm. An amount of 0.16 g of the material was saturated with 10 ml of protein aqueous solution at different concentrations (1 mg·mL⁻¹, 2 mg·mL⁻¹, 4 mg·mL⁻¹, 6 mg·mL⁻¹). The suspensions were incubated in a stirrer (room temperature, 120 rpm) for 24 hours. The amount of adsorbed protein was determined by decrease in optical density at 280 nm in the supernatants. Equilibrium concentration and amount of protein adsorbed to the material were calculated. Desorption experiments were carried out by changing the elution buffer with 1 M NaCl.

### Example 10: Nucleic Acids Purification Analysis Protocol

Preparation of a Bacterial Nucleic Acid Simple for Using DNA-Tip (MINIPREP).

Previously, an E. coli culture in LB medium is prepared in a 125 mL Erlenmeyer.

Centrifuge 2 ml of the E. coli culture at 12000 rpm. Discard the supernatant.

Add 100 μL of Solution 1. Vortex for disintegration of cell pellet.

Add 150 μL of Solution 2. Gently mix by inversion. Incubate not more than 5 min at room temperature.

Add 200 μL of Solution 3. Promptly mix by inversion. Incubate 5 minutes in ice.

Centrifuge 15 min at 12000 rpm. Recover 400 μL from the supernatant (Incubation solution) and transfer to another sterile Eppendorf.

Solution 1: GTE Buffer (50 mM glucose, 25 mM Tris, 10 mM EDTA) pH 8.

Solution 2: 2% SDS and 0.4 N NaOH. Mix in equal parts right before use.

Solution 3: 4.5 M ClGu in 3.0 M sodium acetate buffer pH 4.8.

DNA sample purified by MINIPREP.

Prepare 40 μL of DNA purified with 120 μL of 3M ClGu (Incubation solution).

### Ejemplo 11: Protocol for Use of the Micropipette Tip Bi-Structured Matrix with Silica Nanoparticles (DNA-Tip)

Prepare the Following Solutions:

Incubation solution: DNA simple purified by MINIPREP of Example 10.

Elution solution: Sterile TE buffer (Tris 10 mM—EDTA 1 mM).

Adsorption Procedure

Take 100 μL of the incubation solution using a DNA-Tip in a p200 micropipette.

Incubate for 30 sec at room temperature. Discharge the contents from the DNA-Tip.

Wash the DNA-Tip with 150 μL of 90% isopropanol and discard.

Pull and push the plunger several times for discarding the remaining isopropanol. A sterile Eppendorf may be placed at the tip of the DNA-Tip to prevent contaminations.

Leave to dry 15 min at 37° C. or leave at room temperature until isopropanol has completely evaporated.

Prepare a sterile Eppendorf with 20 μL of sterile elution solution.

Graduate the micropipette in 150 μl. Take 20 μL of the elution solution. Pull and push the plunger several times so that the solution washes the largest possible area in the interior of the DNA-Tip.

Totally discard the contents of the DNA-Tip. Collect the eluate in a 0.5 ml sterile Eppendorf tube and preserve on ice.

Simple analysis is made by means of agarose gel electrophoresis technique and UV-vis spectrophotometry using a Nanodrop 1000 for quantification of purified DNA.

### BIBLIOGRAFY

Bae, K. H., et al., Biotechnol. Progr., 22, 297-302, 2006.
Cabrera, K., et al., J. High Resol. Chromatogr., 23, 93-99, 2000.
Casolaro, M., et al., Biomacromol., 7, 1439-1448, 2006.
Fei, B., et al., J. Appl. Polym. Sci., 78, 278-283, 2000.
Hoffman, A. S., Adv. Drug Deliv. Rev., 54, 3-12, 2002.
IAEA Tecdoc series. Book: Radiation Processed Materials in Products from Polymers for Agricultural Applications, volume: 1745, 2014.
Kobayashi, K., et al., J. Membr. Sci., 76, 209-218, 1993.
Mattiasson et al., book: Macroporous Polymers: Production Properties and Biotechnological/Biomedical Applications. CRC Press. 2009
Park, J.-S., et al., Rad. Phys. Chem., 88, 60-64, 2013.
Przybycien, T. M., et al., Curr. Op. Biotechnol., 15, 469-478, 2004.
Svec, F., J. Chromatogr. A, 1217, 902-924, 2010.
Ventura, A. M., et al., J. Membr. Sci., 321, 350-355, 2008.
Vinogradov, S. V., et al., Adv. Drug Deliv. Rev., 54, 135-147, 2002.
Zhang, M., et al., J. Chromatogr. A, 912, 31-38, 2001.

1-46. (canceled)

47. A bi-structured matrix for solid reactants purification and handling comprising at least a polymeric solid carrier coated with at least one hydrosoluble polymer.

48. The matrix according to claim 47, wherein the solid carrier is selected from the group comprising cross-linked polyurethane foam and micropipette tips.

49. The matrix according to claim 47, wherein the hydrosoluble polymer is selected from the group comprising polyvinyl alcohol, agarose, hydroxyethylcellulose, and combinations thereof.

50. The matrix according to claim 47, further comprising a monomer selected from the group consisting of glycidyl metacrylate monomers (GMA), dimethyl acrylamide (DMAAm), 2-hydroxyethyl metacrylate, metacrylic acid, and combinations thereof.

51. The matrix according to claim 50, wherein the monomer is glycidyl metacrylate (GMA).

52. The matrix according to claim 51, further comprising functional groups selected from the group consisting of sulfonic, iminodiacetic acid (IDA) y ethylenediamine (EDA).

53. The matrix according to claim 47, comprising a cross-linked polyurethane foam solid carrier, coated with a hydrosoluble polymer selected from the group consisting of polyvinyl alcohol, agarose, hydroxyethylcellulose, and glycidyl metacrylate (GMA) monomers bound to said hydrosoluble polymer.

54. The matrix according to claim 47, comprising a cross-linked polyurethane foam solid carrier coated with a hydrosoluble polymer selected from the group consisting of polyvinyl alcohol, agarose, hydroxyethylcellulose, glycidyl metacrylate (GMA) monomers bound to the hydrosoluble polymer and sulfonic groups bound to the glycidyl metacrylate (GMA) monomers.

55. The matrix according to claim 47, comprising a cross-linked polyurethane foam solid carrier coated with a hydrosoluble polymer selected from the group consisting of polyvinyl alcohol, agarose, hydroxyethylcellulose, glycidyl metacrylate (GMA) monomers bound to the hydrosoluble polymer and iminodiacetic acid (IDA) bound to the glycidyl metacrylate (GMA) monomers.

56. The matrix according to claim 47, comprising a pipette tip as a solid carrier internally coated with a hydrosoluble polymer selected from the group consisting of polyvinyl alcohol, agarose and hydroxyethylcellulose.

57. The matrix according to claim 47, comprising a pipette tip as a solid carrier internally coated with a hydrosoluble polymer selected from the group consisting of polyvinyl alcohol, agarose, hydroxyethylcellulose, and silica particles.

58. The matrix according to claim 57, wherein the silica particles have a diameter of between 0.015 and 40 microns.

59. A process for manufacturing a matrix according to claim 47, comprising the following steps:
a) contacting a polymer solid carrier with at least one hydrosoluble polymer until obtaining a polymer solid carrier coated with a hydrosoluble polymer;
b) drying the solid carrier coated with the hydrosoluble polymer and immersing it in an irradiation solution; and
c) irradiating with a source of 60-Cobalt and drying the bi-structured matrix obtained.

60. The process according to claim 59, comprising after step a) a step wherein the solid carrier coated with the hydrosoluble polymer is immersed in a clotting agent selected from the group consisting of 2-propanol, ethanol, 1-propanol, and dioxane.

61. The process according to claim 59, wherein the solid carrier is selected from the group comprising cross-linked polyurethane foam and micropipette tips.

62. The process according to claim 59, wherein the hydrosoluble polymer is selected from the group comprising polyvinyl alcohol, agarose, hydroxyethylcellulose, and combinations thereof.

63. A process for manufacturing the matrix according to claim 53, comprising the following steps:
a) contacting a cross-linked polyurethane foam solid carrier with a hydrosoluble polymer selected from the group consisting of polyvinyl alcohol, agarose, and hydroxyethylcellulose until obtaining a polymer solid carrier coated with a hydrosoluble polymer;
b) immersing the solid carrier coated with the hydrosoluble polymer obtained in the previous step, in a clotting agent selected from the group consisting of 2-propanol, ethanol, 1-propanol, and dioxane;
c) drying the solid carrier coated with the hydrosoluble polymer of the previous step and immersing in an irradiation solution comprising glycidyl metacrylate (GMA) monomers; and
d) irradiating with a source of 60-Cobalt and drying the bi-structured matrix obtained.

64. The process according to claim 63, wherein the irradiation solution comprises ethanol/water.

65. The process according to claim 63, wherein the irradiation solution comprises an amount of between 2% and 6% of the glycidyl metacrylate (GMA) monomer.

66. A process for manufacturing the matrix according to claim 54, comprising the following steps:
a) contacting a cross-linked polyurethane foam solid carrier with a hydrosoluble polymer selected from the group consisting of polyvinyl alcohol, agarose, and hydroxyethylcellulose until obtaining a polymer solid carrier coated with a hydrosoluble polymer;
b) immersing the solid carrier coated with the hydrosoluble polymer obtained in the previous step, in a clotting agent selected from the group consisting of 2-propanol, ethanol, 1-propanol, and dioxane;
c) drying the solid carrier coated with the hydrosoluble polymer of the previous step and immersing it in an irradiation solution comprising glycidyl metacrylate (GMA) monomers;
d) irradiating with a source of 60-Cobalt and drying the bi-structured matrix obtained; and
e) incubating the matrix obtained in the previous step with an aqueous solution comprising sodium sulfite and isopropanol, and drying.

67. The process according to claim 66, wherein the irradiation solution comprises ethanol/water.

68. The process according to claim 66, wherein the irradiation solution comprises an amount of between 2% and 6% of the glycidyl metacrylate (GMA) monomer.

69. A process for manufacturing the matrix according to claim 55, comprising the following steps:
a) contacting a cross-linked polyurethane foam solid carrier with a hydrosoluble polymer selected from the group consisting of polyvinyl alcohol, agarose, and hydroxyethylcellulose until obtaining a polymer solid carrier coated with a hydrosoluble polymer;
b) immersing the solid carrier coated with the hydrosoluble polymer obtained in the previous step, in a clotting agent selected from the group consisting of 2-propanol, ethanol, 1-propanol, and dioxane;
c) drying the solid carrier coated with the hydrosoluble polymer of the previous step and immersing it in an irradiation solution comprising glycidyl metacrylate (GMA) monomers;
d) irradiating with a source of 60-Cobalt and drying the bi-structured matrix obtained;
e) incubating the matrix obtained in the previous step with a solution comprising iminodiacetic acid (IDA) and dimethyl sulfoxide (DMSO); and
f) incubating next in the presence of and acid and drying.

70. The process according to claim 69, wherein the irradiation solution comprises ethanol/water.

71. The process according to claim 69, wherein the irradiation solution comprises an amount of between 2% and 6% of the glycidyl metacrylate (GMA) monomer.

72. A process for manufacturing the matrix according to claim 57, comprising the following steps:
a) contacting a pipette tip as a solid carrier with a suspension containing at least one hydrosoluble polymer and silica particles;
b) drying the pipette tip coated with the hydrosoluble polymer and the silica, and immersing said pipette tip in an irradiation solution; and
c) irradiating with a source of 60-Cobalt and drying the bi-structured matrix obtained.

73. The process according to claim 72, wherein the hydrosoluble polymer is selected from the group comprising polyvinyl alcohol, agarose, hydroxyethylcellulose, and combinations thereof.

74. The process according to claim 72, wherein the irradiation solution comprises ethanol and water.

75. The process according to claim 72, wherein the silica particles have a diameter of between 0.015 and 40 microns.
